(19) United States
(12) Patent Application Publication
(10) Pub. No.: US 2020/0093392 A1
(43) Pub. Date: Mar. 26, 2020

(54) BRAINPRINT SIGNAL RECOGNITION METHOD AND TERMINAL DEVICE

(71) Applicant: Neural FLEX Technology (Shenzhen) CO., LTD, Shenzhen (CN)

(72) Inventors: Yiping Tse, Shenzhen (CN); Zhengqiang Ye, Shenzhen (CN); Hongjie Lu, Shenzhen (CN); Yu Zhang, Shenzhen (CN); Xin Guo, Shenzhen (CN)

(21) Appl. No.: 16/370,856

(22) Filed: Mar. 29, 2019

(30) Foreign Application Priority Data
Sep. 25, 2018 (CN) 201811114765.2

(51) Int. Cl. A61B 5/0476; A61B 5/04; A61B 5/117; A61B 5/00; G16H 50/70

(52) U.S. Cl. CPC A61B 5/0476; A61B 5/04012; A61B 5/117; A61B 5/165; G16H 50/70; A61B 5/0006; A61B 5/7264

(57) ABSTRACT

The present disclosure relates to the field of computer technologies, and provides a brainprint signal recognition method and a terminal device. The method includes: acquiring a brainprint signal to be classified, mapping the brainprint signal to be classified into a vector space, and determining a coefficient vector of the brainprint signal to be classified; acquiring class centers and distance thresholds of respective existing classes in the vector space, where each of the existing classes corresponds to a brainprint signal set; and determining, according to the coefficient vector of the brainprint signal to be classified and the class centers and the distance thresholds of the respective existing classes, the class to which the brainprint signal to be classified belongs.

### CROSS-REFERENCES TO RELATED APPLICATION

This application claims priority to Chinese Patent Application 201811114765.2, filed on Sep. 25, 2018, the content of which is incorporated herein by reference in its entirety.

### TECHNICAL FIELD

The present disclosure relates to the field of computer technologies, and in particular, to a brainprint signal recognition method and a terminal device.

### BACKGROUND

Brainprint is a feature of the brain electrical signal produced by the brain, which is unique, collectable and durable and can be used for identity recognition and verification. There are broad application scenarios for brainprint-based identity recognition. For example, it can be used to compare two or more generated under the same situation or stimulus to determine whether the brainprints are generated from the same person, or whether the brainprints belong to a plurality of classes, where each person's brainprint corresponds to one class. For another example, it can be used to detect whether a driver is in abnormal situations such as drunk driving, taking drugs, or the like. There are two classes (normal and abnormal), that is, the brainprints of normal persons correspond to one class, and the brainprints of abnormal persons correspond to another class. The brainprint signals include resting-state EEG signals, visual evoked potential (VEP) signals, motion imaginary EEG signals, and event related potential signals.

In The traditional brainprint-based identity recognition method, the brainprint is mapped into a vector space, and the generated vector is used to perform machine learning training so as to obtain a classifier to recognize the identity. However, the number of the brainprint signal samples is usually small, for example, when multiple class identity recognition is required, sometimes one class has only one signal, which results in low recognition accuracy of the classifier and cannot guarantee classification performance.

### SUMMARY

In view of this, according to embodiments of the present disclosure, it is provided a brainprint signal recognition method and a terminal device, aiming to solve the problem of traditional brain signal recognition method that the recognition accuracy is low when the number of brain signal samples is small.

According to a first aspect of the prevent disclosure, it is provided a brainprint signal recognition method which includes:

acquiring a brainprint signal to be classified, mapping the brainprint signal to be classified into a vector space, and determining a coefficient vector of the brainprint signal to be classified;

acquiring class centers and distance thresholds of respective existing classes in the vector space, where each of the existing classes corresponds to a brainprint signal set; and

determining, according to the coefficient vector of the brainprint signal to be classified and the class centers and the distance thresholds of the respective existing classes, the class to which the brainprint signal to be classified belongs.

According to a second aspect of the prevent disclosure, it is provided a terminal device comprising a memory, a processor, and a computer program stored in the memory and operable in the processor, wherein the processor is configured to execute the computer program to implement steps of the brainprint signal recognition method according to the first aspect.

According to a third aspect of the prevent disclosure, it is provided a computer readable storage medium with a computer program stored therein, wherein when the computer program is executed by a processor steps of the brainprint signal recognition method according to the first aspect are implemented

The beneficial effects of an embodiment of the present disclosure compared with the prior art include: by mapping the brainprint signal to be classified into a vector space, acquiring a coefficient vector of the brainprint signal to be classified, and then determining, according to the coefficient vector of the brainprint signal to be classified and the class centers and the distance thresholds of the existing classes in the vector space, the class to which the brain pattern signal to be classified belongs, the recognition of the brain pattern signal to be classified can be realized. In an embodiment of the present disclosure the brainprint signal is classified according to the class centers and the distance thresholds of the existing classes in the vector space, the brainprint signal can be accurately recognized even when the number of the brainprint signal samples is small, and recognition accuracy of the brainprint signal can be improved.

### BRIEF DESCRIPTION OF THE DRAWINGS

In order to more clearly illustrate the technical solutions in the embodiments of the present disclosure, the drawings used in the description to the embodiments or the prior art will be briefly described below. It is obvious that the drawings in the following description are just for some embodiments of the present disclosure, those skilled in the art can also obtain other drawings based on these drawings without paying any creative effort.

FIG. 1 is a flowchart of an implementation of a brainprint signal recognition method according to an embodiment of the present disclosure;

FIG. 2 is a flowchart of implementing of establishing a vector space in a brainprint signal recognition method according to an embodiment of the present disclosure;

FIG. 3 is a flowchart of an implementation of determining class centers and distance thresholds of respective existing classes in a brainprint signal recognition method according to an embodiment of the present disclosure;

FIG. 4 is a flowchart of an implementation of determining distance thresholds of respective existing classes according to third distance values in a brainprint signal recognition method according to an embodiment of the present disclosure;

FIG. 5 is a flowchart of an implementation of determining a class to which a brainprint signal to be classified belongs in a brainprint signal recognition method according to an embodiment of the present disclosure;

FIG. 6 is a flowchart of an implementation of determining whether a brainprint signal to be classified belongs to a class in existing classes in a brainprint signal recognition method according to an embodiment of the present disclosure;

FIG. 7 is a flowchart of an implementation of determining distance thresholds of respective second classes in a brainprint signal recognition method according to an embodiment of the present disclosure;

FIG. 8 is a flowchart of an implementation of determining distance thresholds of respective second classes according to second distance values in a brainprint signal recognition method according to an embodiment of the present disclosure;

FIG. 9 is a schematic diagram of a brainprint signal recognition apparatus according to an embodiment of the present disclosure; and

FIG. 10 is a schematic diagram of a terminal device according to an embodiment of the present disclosure.

### DESCRIPTION OF THE EMBODIMENTS

In the following description, in order to describe but not intended to limit, concrete details such as specific system structure, technique, and so on are proposed, thereby facilitating comprehensive understanding of the embodiments of the present application. However, it will be apparent to the ordinarily skilled one in the art that, the present application can also be implemented in some other embodiments without these concrete details. In some other conditions, detailed explanations of method, circuit, device and system well known to the public are omitted, so that unnecessary details can be prevented from obstructing the description of the present application.

In order to explain the technical solutions described in the present disclosure, the following description will be made by way of specific embodiments.

FIG. 1 is a flowchart of a brainprint signal recognition method according to an embodiment of the present disclosure. The method will be described in detail as follows.

In S101, a brainprint signal to be classified is acquired and mapped into a vector space, and a coefficient vector of the brainprint signal to be classified is determined.

In this embodiment, the execution entity may be a terminal device, such as a desktop computer, a laptop, a palmtop computer, a mobile phone, and a cloud server, etc. The brainprint signal to be classified is a brainprint signal that needs to be recognized. The brainprint signal to be classified may be acquired from a brain wave acquisition device.

For example, the brain wave acquisition device acquires the brainprint signal of the subject, sends the brainprint signal directly to a server or sends the brainprint signal to the server through a mobile device such as a mobile phone or a tablet computer, and the server recognizes the brainprint signal; or the brain wave acquisition device sends the brainprint signal to the mobile device, and the mobile device recongnizes the brainprint signal and sends the recognition result of the brainprint signal to the server for storage.

The vector space may be a pre-established vector space. The brainprint signal to be classified may be mapped to the vector space by performing a transform on the brainprint signal to be classified. The transform includes, but is not limited to, a Fourier transform, a wavelet transform, a Hilbert transform, and a Taylor series transform and so on, which are not limited herein. Different brainprint signals will have different coefficients in the same vector space. The coefficient vector of the brainprint signal to be classified is a vector formed for the coefficient of the brainprint signal to be classified in the vector space.

In S102, class centers and distance thresholds of respective existing classes in the vector space are acquired, where each of the existing classes corresponds to a brainprint signal set.

In this embodiment, the brainprint signals belonging to a same class form a brainprint signal set, and each brainprint signal set corresponds to an existing class. Each brainprint signal set includes at least one brainprint signal sample. The existing classes are classes that currently exist. For example, currently there are two brainprint signal sets, correspondingly there are two classes, and the two classes are two existing classes. In the process of the brainprint signal recognition, the total number of the existing classes may remain unchanged or may be increased. For example, when there is a new class during the recognition process, the new class is an added existing class for the next recognition and the total number of the existing classes is increased by 1.

For example, in the situation of detecting whether the subject is drunk driving, the class of the brainprint signal may be a drunk driving class and a non-drunk driving class, and there are two existing classes. In this case two existing classes have been able to complete the identity recognition purpose. Therefore, a new existing class will not be added during the identity recognition process. In the situation of determining whether two or more brainprints generated under the same situation/stimulus are generated by the same person, the classes of the brainprints may be the persons corresponding to the brainprint signals, and in the recognition process if a brainprint signal is not from a person corresponding to an existing class, a new class may be added as the class to which this brainprint signal belongs, and in this situation the total number of existing classes may be increased with the recognition process.

Each existing class corresponds to a class center. The class center is a center of vectors representing the coefficient vectors of the brainprint signals corresponding to the existing class. The class center may be expressed as a vector, a set of coefficients, a coordinate value, or the like, which is not limited herein. Each existing class corresponds to a distance threshold. The distance threshold may be used as a reference value for determining whether a brainprint signal belongs to an existing class. The class center and distance threshold of the existing class are used to identify and classify the brainprint signals.

In S103, the class to which the brainprint signal to be classified belongs is determined according to the coefficient vector of the brainprint signal to be classified and the class centers and the distance thresholds of respective existing classes.

In this embodiment, the brainprint signal to be classified may be identified according to the class centers and the distance thresholds of the respective existing classes and the coefficient vector of the brainprint signal to be classified to determine the class to which the brainprint signal to be classified belongs. The brainprint signal to be classified may belong to a class in the current existing classes, or may belong to a class other than the currently existing classes, which is not limited herein.

In an embodiment of the present disclosure, by mapping the brainprint signal to be classified into a vector space, acquiring a coefficient vector of the brainprint signal to be classified, and then determining, according to the coefficient vector of the brainprint signal to be classified and the class centers and the distance thresholds of the existing classes in the vector space, the class to which the brain pattern signal to be classified belongs, the recognition of the brain pattern signal to be classified can be realized. In an embodiment of the present disclosure the brainprint signal is classified according to the class centers and the distance thresholds of the existing classes in the vector space, the brainprint signal can be accurately recognized even when the number of the brainprint signal samples is small, and recognition accuracy of the brainprint signal can be improved.

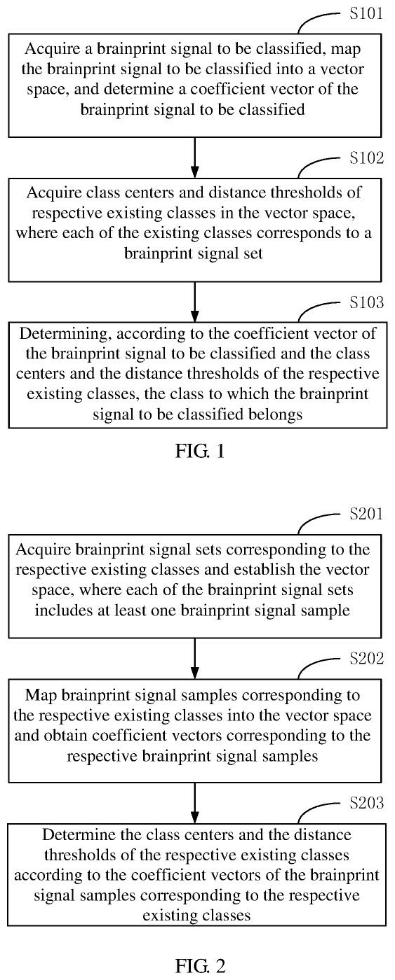

As an embodiment of the present disclosure, as shown in FIG. 2, before the step S101, the foregoing method may further include:

In S201, brainprint signal sets corresponding to the respective existing classes is acquired and the vector space is established, where each of the brainprint signal sets includes at least one brainprint signal sample.

In this embodiment, before recognizing the brainprint signal to be classified, the vector space may be established in advance, and the class centers and the distance thresholds of the respective existing classes in the vector space may be determined. When the vector space is established, a preset value may be used as the basis of the vector space, and the coefficient obtained by transforming an existing brainprint signal may be used as the basis of the vector space, which is not limited herein.

In S202, the brainprint signal samples corresponding to the respective existing classes are mapped into the vector space, and coefficient vectors corresponding to the respective brainprint signal samples are obtained.

In this embodiment, a brainprint signal sample corresponding to an existing class is a brainprint signal sample included in the brainprint signal set corresponding to the existing class. The coefficient vector of the brainprint signal sample corresponding to the existing class may be obtained by transforming the brainprint signal sample corresponding to the existing class to map it into the vector space. The transform includes, but is not limited to, a Fourier transform, a wavelet transform, a Hilbert transform, a Taylor series transform and so on, and may be determined according to actual needs, which is not limited herein.

In S203, the class centers and the distance thresholds of the respective existing classes are determined according to the coefficient vectors of the brainprint signal samples corresponding to the respective existing classes.

In this embodiment, the class center and the distance threshold of an existing class may be determined according to the coefficient vectors of the brainprint signal samples corresponding to the existing class.

Optionally, the class center of any of the existing classes is an operation result of the coefficient vectors of the respective brain signal samples corresponding to the any of the existing classes.

In this embodiment, the class center of an existing class is the operation result of the coefficient vectors of the respective brainprint signal samples corresponding to the existing class. If the brainprint signal set corresponding to the existing class contains only one brainprint signal sample, the class center of the existing class is the coefficient or the coefficient vector of the brainprint signal sample. If two or more brainprint signal samples are included in the brainprint signal set corresponding to the existing class, the class center of the existing class is the operation result of the coefficient vectors of the brainprint signal samples. The operation result refers to the result of performing mathematical operation on these coefficient vectors. The mathematical operation may be a mathematical operation (such as multiplication, addition, etc.), or a combination of mathematical operations, and the like, which is not limited herein.

Optionally, the class center of any of the existing classes is a vector with the smallest distance average to the coefficient vectors of the respective brain signal samples corresponding to the any of the existing classes in the vector space.

In this embodiment, a vector with the smallest distance average to the coefficient vectors of the respective brainprint signal samples corresponding to an existing class in the vector space may be selected as the class center of the existing class. For example, the coefficient vectors of the respective brainprint signal samples corresponding to the existing class may be directly mathematically operated to obtain a vector with the smallest distance average value to the respective coefficient vectors; or multiple candidate vectors are selected in the vector space, distance average values of the respective candidate vectors to the coefficient vectors of the respective brainprint signal samples corresponding to an existing class are respectively calculated, and the candidate vector with the smallest distance average is selected from the plurality of candidate vectors as the class center of the existing class. It should be noted that there are other methods for obtaining the vector with the smallest distance average to the coefficient vectors of the respective brainprint signal samples, which are not limited herein.

In this embodiment, by taking the vector with the smallest distance average to the coefficient vectors of the respective brainprint signal samples corresponding to an existing class in the vector space as the class center of the existing class, the determined class center can more accurately reflect the center of the brainprint signal set correspondting to the existing class, thereby improving the recognition accuracy.

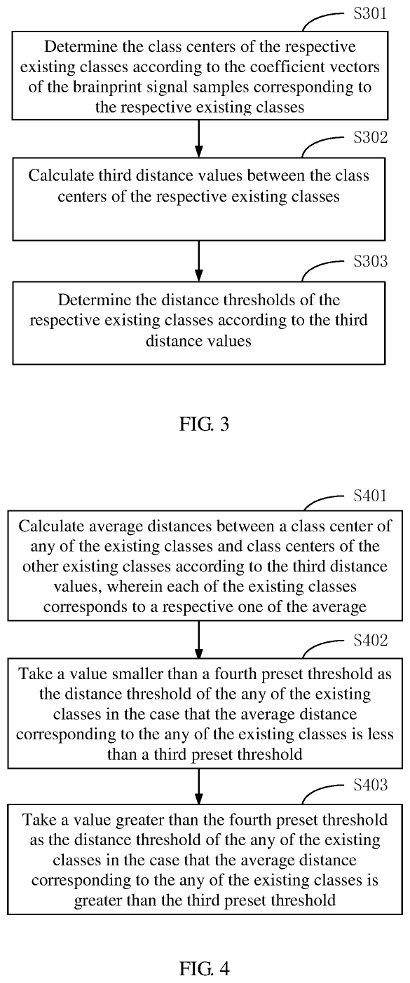

As an embodiment of the present disclosure, as shown in FIG. 3, S203 may include:

In S301, the class centers of the respective existing classes are determined according to the coefficient vectors of the brainprint signal samples corresponding to the respective existing classes.

In this embodiment, the class center of an existing class may be determined according to the coefficient vectors of the brainprint signal samples corresponding to the existing class.

In S302, third distance values between the class centers of the respective existing classes are calculated.

In this embodiment, the distance between the class centers of any two existing classes may be respectively calculated according to the class centers of the respective existing classes to obtain the third distance values between the class centers of the respective existing classes. For example, there are three existing classes A, B, and C, and the third distance values may include the distance value between the class center of A and the class center of B, the distance value between the class center of A and the class center of C, and the distance value between the class center of B and the class center of C.

In S303, the distance thresholds of the respective existing classes are determined according to the third distance values.

In this embodiment, the distance thresholds of the respective existing classes may be determined according to the third distance values calculated in S302.

As an embodiment of the present disclosure, as shown in FIG. 4, S303 may include:

In S401, average distances between a class center of any of the existing classes and class centers of the other existing classes are calculated according to the third distance values, where each of the existing classes corresponds to a respective one of the average distances.

In this embodiment, the average distance between the class center of an existing class and the class centers of the other existing classes may be used as the average distance corresponding to the existing class. For example, there are three existing class A, B, and C. The distance value between the class center of A and the class center of B is AB, the distance value between the class center of A and the class center of C is AC, and the average distance corresponding to A is the average of the two values AB and AC.

In S402, a value smaller than a fourth preset threshold is taken as the distance threshold of the any of the existing classes in the case that the average distance corresponding to the any of the existing classes is less than a third preset thresholds.

In this embodiment, if the average distance corresponding to an existing class is less than the third preset threshold, the distance threshold of the existing class is determined to be a value smaller than the fourth preset threshold.

In S403, a value greater than the fourth preset threshold is taken as the distance threshold of the any of the existing classes in the case that the average distance corresponding to the any of the existing classes is greater than the third preset threshold.

In this embodiment, if the average distance corresponding to an existing class is greater than the third preset threshold, the distance threshold of the existing class is determined to be a value greater than the fourth preset threshold.

Optionally, the third preset threshold is an average of the third distance values between the class centers of the respective existing classes.

In this embodiment, the average value of the distance values between the class centers of the respective existing classes may be used as the third preset threshold. For example, there are three existing classes A, B, and C. The distance between the class center of A and the class center of B is AB, the distance between the class center of A and the class center of C is AC, the distance between the class center of B and the class center of C is BC, and the third preset threshold is the average of AB, AC, and BC.

In this embodiment, by using the average value of the third distance values between the class centers of the respective existing classes as the third preset threshold, and comparing the third preset threshold with the average distance corresponding to an existing class to determine the distance threshold of the existing class, it is possible to set a more appropriate distance threshold for the existing class according to the comparison result, thereby making the classification of the brain signal more accurate. For example, the class A is far away from other classes, which means that the surrounding area of the class A is relatively empty. At this time, the average distance corresponding to the class A is greater than the average of the third distance values between all classes, so a larger value (such as a value greater than the fourth preset threshold) is taken as the distance threshold of the class A. The class B is closer to other classes, which means that the surrounding area of the class B is relatively dense. At this time, the average distance corresponding to the class B is less than the average of the third distance values between all classes, so a smaller value (such as a value smaller than the fourth preset threshold) is taken as the distance threshold of the class A. In this way, when determining the distance threshold of a class, the class distribution situation (dense or sparse, etc.) around the class can be fully considered, so that the setting of the distance threshold is more reasonable and the classification accuracy can be improved.

Optionally, each existing class corresponds to a fourth preset threshold, and the fourth preset threshold of any of the existing classes is a product of an average distance corresponding to the any of the existing classes and a preset coefficient, wherein the preset coefficient is greater than 0 and less than or equal to 1.

In this embodiment, the product of the average distance corresponding to an existing class and the preset coefficient may be used as the fourth preset threshold of the existing class. For example, if the average distance corresponding to an existing class is a, then a*m may be used as the fourth preset threshold of the existing class, where 0<m≤1.

In this embodiment, by using the product of the average distance corresponding to an existing class and the preset coefficient as the fourth preset threshold of the existing class, the fourth preset threshold can be adjusted by the preset coefficient, thereby adjusting the distance threshold of the existing class, which can improve the applicability and accuracy of the recognition method according to the embodiment. For example, if a class is far away from other samples, it means that the surrounding area of the class is relatively empty. At this time, if the average distance corresponding to the class is directly used as the distance threshold of the class for classification and determination, actual existing new class may be concealed. In this embodiment, by multiplying a preset coefficient, the distance threshold of the class can be adjusted so that that the situation that the signal sample belonging to the new class is covered due to the distance threshold of the class is too large can be avoided, thereby making the classification more accurate.

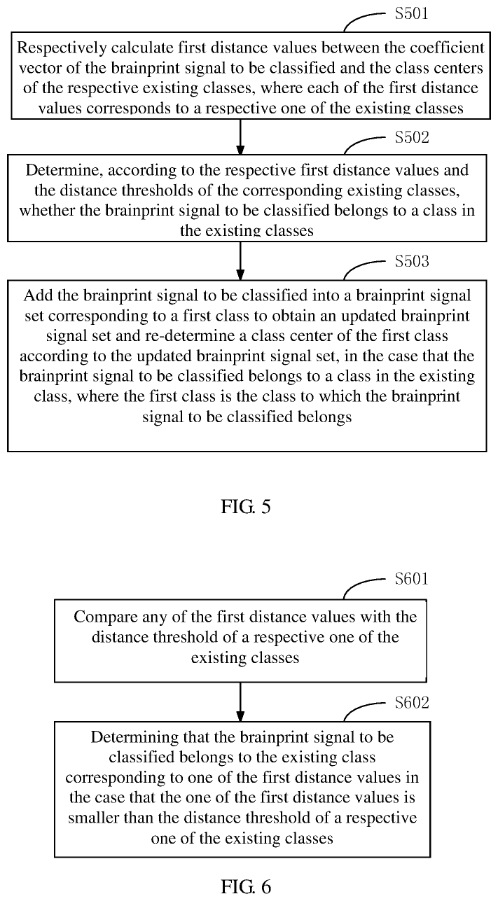

As an embodiment of the present disclosure, as shown in FIG. 5, S103 may include:

In S501, first distance values between the coefficient vector of the brainprint signal to be classified and the class centers of the respective existing classes are respectively calculated, where each of the first distance values corresponds to a respective one of the existing classes.

In S502, it is determined, according to the respective first distance values and the distance thresholds of the corresponding existing classes, whether the brainprint signal to be classified belongs to a class in the existing classes.

In this embodiment, the first distance values between the coefficient vector of the brainprint signal to be classified and the class centers of the respective existing classes may be calculated, and then it is determined, according to the respective first distance values and the distance thresholds of the corresponding existing classes, whether the brainprint signal to be classified belongs to a class in the existing classes.

As an embodiment of the present disclosure, as shown in FIG. 6, S502 may include:

In S601, any of the first distance values is compared with the distance threshold of a respective one of the existing classes.

In S602, in the case that one of the first distance values is smaller than the distance threshold of a respective one of the existing classes, it is determined that the brainprint signal to be classified belongs to the existing class corresponding to the one of the first distance values.

In this embodiment, a first distance value of the brainprint signal to be classified may be compared with a distance threshold of the existing class corresponding to the first distance value. If the first distance value is smaller than the distance threshold of a respective existing class, it is determined that the brainprint signal to be classified belongs to the existing class corresponding to the first distance values.

In S503, the brainprint signal to be classified is added into a brainprint signal set corresponding to a first class to obtain an updated brainprint signal set and a class center of the first class is re-determined according to the updated brainprint signal set in the case that the brainprint signal to be classified belongs to a class in the existing class, where the first class is the class to which the brainprint signal to be classified belongs.

In this embodiment, if the brainprint signal to be classified belongs to a class in the existing classes, the class is taken as the first class, and the brainprint signal to be classified is added into the brainprint signal set corresponding to the first class to obtain the updated brainprint signal set. And the class center of the first class is re-determined based on the updated brainprint signal set.

In this embodiment, after it is determined that the brainprint signal to be classified belongs to a class in the existing classes, the brainprint signal to be classified is added into the brainprint signal set corresponding to the class, and the class center of the class is re-determined.

Optionally, the process of re-determining the class center of the class may include:

selecting a plurality of candidate vectors (for example, obtaining a plurality of candidate vectors by randomly adjusting respective coefficients corresponding to the original class center, etc.) in a preset area near the original class center of the class to which the brainprint signal to be classified belongs, respectively calculating the average of the distances between the original class center and the candidate vectors and the coefficient vectors of the respective brain signals (including the brainprint signal to be classified) corresponding to the class, and taking the vector with the smallest average distance in the original class center and all candidate vectors as the class center of the class with the brainprint signal to be classified being added.

The addition of a brainprint signal to be classified generally has small influence on the class center of the class, that is, the re-determined class center of the class is usually near the original class center. Therefore, in the present embodiment, by selecting a plurality of candidate vectors in an area near the original class center of the class to which the brainprint signal to be classified belongs and taking the vector with the smallest average distance in the original class center and all candidate vectors as the class center of the class with the brainprint signal to be classified being added, the class center of the class can be quickly and accurately re-determined, the determination speed of the class center and the recognition efficiency can be improved.

As an embodiment of the present disclosure, after S502, the foregoing method may further include:

creating an added class and determining a class center of the added class according to the coefficient vector of the brainprint signal to be classified in the case that the brainprint signal to be classified does not belong to a class in the existing classes.

In this embodiment, after determining that the brainprint signal to be classified does not belong to the class in the existing classes, a newly added class is created, and the brainprint signal to be classified is added to the brainprint signal set corresponding to the new class. At this time, the brainprint signal set corresponding to the newly added class only includes the brainprint signal to be classified, the class center of the newly added class may be determined according to the coefficient vector of the brainprint signal to be classified. For example, the newly added class only includes the brainprint signal to be classified, and the coefficient vector of the brainprint signal to be classified can be directly used as the center of the newly added class.

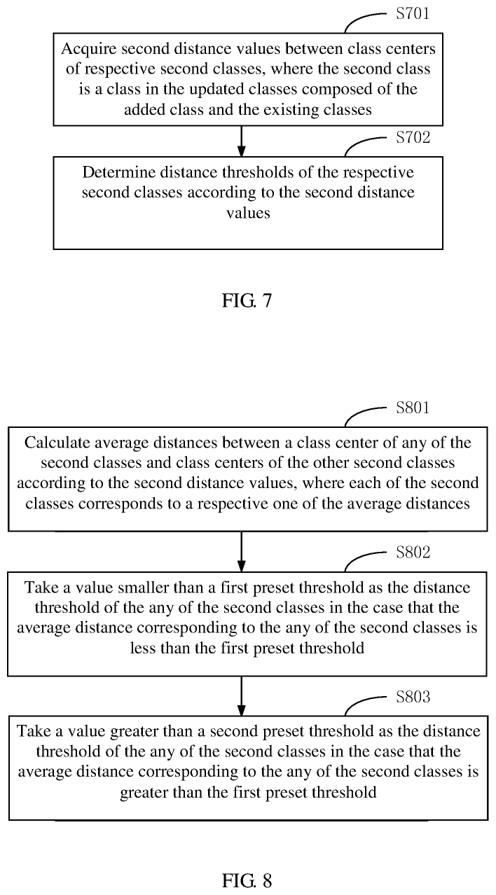

As an embodiment of the present disclosure, as shown in FIG. 7, after the step of creating an added class and determining a class center of the added class according to the coefficient vector of the brainprint signal to be classified in the case that the brainprint signal to be classified does not belong to a class in the existing classes, the foregoing method may further include:

In S701, second distance values between class centers of respective second classes are acquired, where the second class is a class in the updated classes composed of the added class and the existing classes.

In S702, distance thresholds of the respective second classes are determined according to the second distance values.

In this embodiment, a newly added class can be added to the existing classes to form updated classes. The updated classes include new class and original existing classes. In the next brainprint signal recognition process, all classes in the updated classes are used as existing classes to recognize the next brainprint signal to be classified.

After determining the class center of the newly added class, the distance thresholds of respective classes in the updated classes may be recalculated. For the convenience of description, the classes in the updated classes are referred to as the second class, that is, the original existing classes are second class, and the newly added class is also a second class. For example, the original existing classes are A, B, C, and D, the added class is E, then the updated classes include A, B, C, D, and E, all of which are referred to as the second class.

The distance between the class centers of any two second classes may be respectively calculated according to the class centers of the respective second classes, the second distance values between the class centers of the respective second classes are obtained, and then the distance thresholds of the respective second classes are determined according to the calculated second distance values.

As an embodiment of the present disclosure, as shown in FIG. 8, S702 may include:

In S801, average distances between a class center of any of the second classes and class centers of the other second classes are calculated according to the second distance values, where each of the second classes corresponds to a respective one of the average distances.

In S802, a value smaller than a first preset threshold is taken as the distance threshold of the any of the second classes in the case that the average distance corresponding to the any of the second classes is less than the first preset threshold.

In S803, a value greater than a second preset threshold is taken as the distance threshold of the any of the second classes in the case that the average distance corresponding to the any of the second classes is greater than the first preset threshold.

In this embodiment, the method of determining the distance thresholds in S801 to S803 is the same as the method for determining the distance thresholds in S401 to S403 as described above, and the beneficial effects are the same, so it will be only briefly described.

The specific determination method is as follows.

The average distance between the class center of one second class and the class centers of the other second classes may be taken as the average distance corresponding to the second class. If the average distance corresponding to the second class is less than the first preset threshold, the distance threshold of the second class is determined to be a value smaller than the second preset threshold. If the average distance corresponding to the second class is greater than the first preset threshold, the distance threshold of the second class is determined to be a value greater than the second preset threshold.

Optionally, the first preset threshold is an average value of the second distance values between the class centers of the respective second classes.

In this embodiment, the average value of the distance values between the class centers of the respective second classes may be used as the first preset threshold.

Optionally, each of the second classes corresponds to a second preset threshold, and the second preset threshold of the any of the second classes is a product of an average distance corresponding to the any of the second classes and a preset coefficient, wherein the preset coefficient is greater than 0 and less than or equal to 1.

In this embodiment, the product of the average distance corresponding to a second class and the preset coefficient may be used as the second preset threshold of the second class.

In this embodiment of the present disclosure, when a sample is added, a new sample can be quickly added into the existing classes or a class for the new sample can be quickly established, and by adjusting the distance threshold the recognition of the brainprint signal can be more accurate.

In the embodiment of the present disclosure, by mapping the brainprint signal to be classified into a vector space, acquiring a coefficient vector of the brainprint signal to be classified, and then determining, according to the coefficient vector of the brainprint signal to be classified and the class centers and the distance thresholds of the existing classes in the vector space, the class to which the brain pattern signal to be classified belongs, the recognition of the brain pattern signal to be classified can be realized. In an embodiment of the present disclosure the brainprint signal is classified according to the class centers and the distance thresholds of the existing classes in the vector space, the brainprint signal can be accurately recognized even when the number of the brainprint signal samples is small, and recognition accuracy of the brainprint signal can be improved.

It should be understood that, values of serial numbers of the steps in the above embodiments don't mean the execution sequence of the steps, the execution sequence of the steps should be determined by its function and internal logics, and should not be construed as limiting the implementation process of the embodiments of the present application.

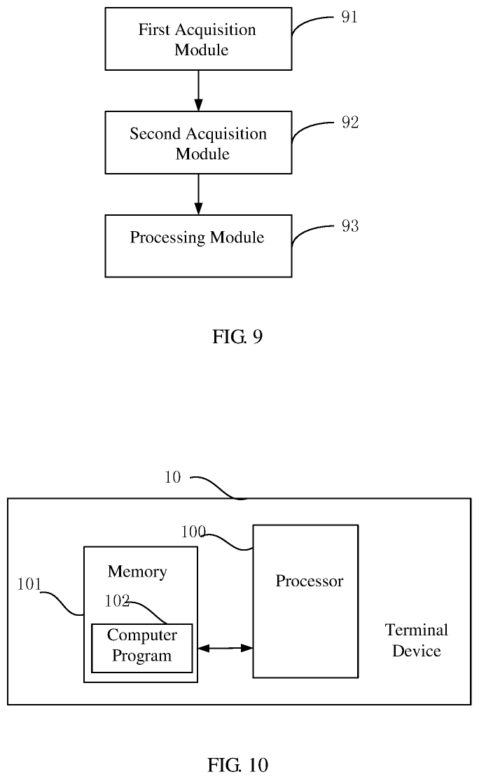

Corresponding to the brainprint signal recognition method described in the above embodiments, FIG. 9 is a schematic diagram of a brainprint signal recognition apparatus according to an embodiment of the present disclosure. For the convenience of explanation, only the parts related to the present embodiment are shown.

Referring to FIG. 9, the apparatus includes a first acquisition module 91, a second acquisition module 92, and a processing module 93.

The first acquisition module 91 is configured to acquire a brainprint signal to be classified, to map the brainprint signal to be classified into a vector space, and to determine a coefficient vector of the brainprint signal to be classified.

The second acquisition module 92 is configured to acquire class centers and distance thresholds of respective existing classes in the vector space, where each of the existing classes corresponds to a brainprint signal set;

The processing module 93 is configured to determine, according to the coefficient vector of the brainprint signal to be classified and the class centers and the distance thresholds of the respective existing classes, the class to which the brainprint signal to be classified belongs.

Optionally, the processing module 93 is configured to:

respectively calculate first distance values between the coefficient vector of the brainprint signal to be classified and the class centers of the respective existing classes, where each of the first distance values corresponds to a respective one of the existing classes;

determine, according to the respective first distance values and the distance thresholds of the corresponding existing classes, whether the brainprint signal to be classified belongs to a class in the existing classes; and

add the brainprint signal to be classified into a brainprint signal set corresponding to a first class to obtain an updated brainprint signal set and re-determine a class center of the first class according to the updated brainprint signal set, in the case that the brainprint signal to be classified belongs to a class in the existing class, wherein the first class is the class to which the brainprint signal to be classified belongs.

Optionally, the processing module 93 is configured to:

compare any of the first distance values with the distance threshold of a respective one of the existing classes; and

in the case that one of the first distance values is smaller than the distance threshold of a respective one of the existing classes, determine that the brainprint signal to be classified belongs to the existing class corresponding to the one of the first distance values.

Optionally, the processing module 93 is configured to:

create an added class and determine a class center of the added class according to the coefficient vector of the brainprint signal to be classified in the case that the brainprint signal to be classified does not belong to a class in the existing classes

Optionally, the processing module 93 is configured to:

acquire second distance values between class centers of respective second classes, where the second class is a class in the updated classes composed of the added class and the existing classes; and

determine distance thresholds of the respective second classes according to the second distance values.

Optionally, the processing module 93 is configured to:

calculate average distances between a class center of any of the second classes and class centers of the other second classes according to the second distance values, where each of the second classes corresponds to a respective one of the average distances;

take a value smaller than a first preset threshold as the distance threshold of the any of the second classes in the case that the average distance corresponding to the any of the second classes is less than the first preset threshold; and

take a value greater than a second preset threshold as the distance threshold of the any of the second classes in the case that the average distance corresponding to the any of the second classes is greater than the first preset threshold.

Optionally, the first preset threshold is an average value of the second distance values between the class centers of the respective second classes.

Optionally, each of the second classes corresponds to a second preset threshold, and the second preset threshold of the any of the second classes is a product of an average distance corresponding to the any of the second classes and a preset coefficient, wherein the preset coefficient is greater than 0 and less than or equal to 1.

Optionally, the apparatus further includes an establishing module, the establishing module is configured to:

acquire brainprint signal sets corresponding to the respective existing classes and establish the vector space, where each of the brainprint signal sets includes at least one brainprint signal sample;

map brainprint signal samples corresponding to the respective existing classes into the vector space and obtain coefficient vectors corresponding to the respective brainprint signal samples; and

determine the class centers and the distance thresholds of the respective existing classes according to the coefficient vectors of the brainprint signal samples corresponding to the respective existing classes.

Optionally, the class center of any of the existing classes is an operation result of the coefficient vectors of the respective brain signal samples corresponding to the any of the existing classes.

Optionally, the class center of any of the existing classes is a vector with the smallest average distance to the coefficient vectors of the respective brain signal samples corresponding to the any of the existing classes in the vector space.

Optionally, the establishing module is configured to:

determine the class centers of the respective existing classes according to the coefficient vectors of the brainprint signal samples corresponding to the respective existing classes;

calculate third distance values between the class centers of the respective existing classes; and

determine the distance thresholds of the respective existing classes according to the third distance values.

Optionally, the establishing module is configured to:

calculate average distances between a class center of any of the existing classes and class centers of the other existing classes according to the third distance values, where each of the existing classes corresponds to a respective one of the average distances;

take a value smaller than a fourth preset threshold as the distance threshold of the any of the existing classes in the case that the average distance corresponding to the any of the existing classes is less than a third preset threshold; and

take a value greater than the fourth preset threshold as the distance threshold of the any of the existing classes in the case that the average distance corresponding to the any of the existing classes is greater than the third preset threshold.

In an embodiment of the present disclosure, by mapping the brainprint signal to be classified into a vector space, acquiring a coefficient vector of the brainprint signal to be classified, and then determining, according to the coefficient vector of the brainprint signal to be classified and the class centers and the distance thresholds of the existing classes in the vector space, the class to which the brain pattern signal to be classified belongs, the recognition of the brain pattern signal to be classified can be realized. In an embodiment of the present disclosure the brainprint signal is classified according to the class centers and the distance thresholds of the existing classes in the vector space, the brainprint signal can be accurately recognized even when the number of the brainprint signal samples is small, and recognition accuracy of the brainprint signal can be improved.

FIG. 10 is a schematic diagram of a terminal device according to an embodiment of the present disclosure. As shown in FIG. 10, the terminal device 10 of this embodiment includes a processor 100, a memory 101, and a computer program 102, such as a program, stored in the memory 101 and operable in the processor 100. The processor 100 is configured to execute the computer program 102 to implement the steps in each method embodiment described above, such as steps 101 to 103 shown in FIG. 1. Alternatively, the processor 100 is configured to execute the computer program 102 to implement the functions of the modules/units in each device embodiment described above, such as the functions of the modules 91 to 93 shown in FIG. 9.

Exemplarily, the computer program 102 can be divided into one or a plurality of modules/units, the one or plurality of modules/units are stored in the memory 101, and executed by the processor 100 so as to implement the present application. The one or plurality of modules/units can be a series of computer program instruction segments that can accomplish particular functionalities, these instruction segments are used for describing an executive process of the computer program 102 in the terminal device 10.

The terminal device 10 may be a computing device such as a desktop computer, a laptop, a palmtop computer, and a cloud server. The terminal device may include, but is not limited to, the processor 100 and the memory 101. It will be understood by those skilled in the art that FIG. 10 is merely an example of the terminal device 10, does not constitute a limitation of the terminal device 10, may include more or less components than those illustrated, or may combine some components, or may include different components. For example, the terminal device may further include an input/output device, a network access device, a bus, a display, and the like.

The so called processor 100 may be CPU (Central Processing Unit), and may alternatively be other general purpose processor, DSP (Digital Signal Processor), ASIC (Application Specific Integrated Circuit), FGPA (Field-Programmable Gate Array), or some other programmable logic devices, discrete gate or transistor logic device, discrete hardware component, etc. The general purpose processor may be a microprocessor, or alternatively, the processor may alternatively be any conventional processor or the like.

The memory 101 may be an internal storage unit of the terminal device 10, such as a hard disk or an internal storage unit of the terminal device 10. The storage device 101 may alternatively be an external storage device of the terminal device 10, such as a plug-in hard disk, a SMC (Smart Media Card), a SD (Secure Digital) card, a FC (Flash Card) or the like, equipped on the terminal device 10. Further, the memory 101 may include both the internal storage unit and the external storage device of the terminal device 10. The memory 101 is configured to store the computer programs, and other procedures and data needed by the terminal device 10 for determining wellbore cross-sectional shape. The memory 101 can also be configured to store data that has been output or being ready to be output temporarily.

It can be clearly understood by the one of ordinary skill in the art that, for describing conveniently and concisely, dividing of the aforesaid various functional units, functional modules is described exemplarily merely, in an actual application, the aforesaid functions can be assigned to different functional units and functional modules to be accomplished, that is, an inner structure of a data synchronizing device is divided into functional units or modules so as to accomplish the whole or a part of functionalities described above. The various functional units, modules in the embodiments can be integrated into a processing unit, or each of the units exists independently and physically, or two or more than two of the units are integrated into a single unit. The aforesaid integrated unit can by either actualized in the form of hardware or in the form of software functional units. In addition, specific names of the various functional units and modules are only used for distinguishing from each other conveniently, but not intended to limit the protection scope of the present application. Regarding a specific working process of the units and modules in the aforesaid device, reference can be made to a corresponding process in the aforesaid method embodiment, it is not repeatedly described herein.

In the aforesaid embodiments, the description of each of the embodiments is emphasized respectively, regarding a part of one embodiment which isn't described or disclosed in detail, please refer to relevant descriptions in some other embodiments.

The ordinarily skilled one in the art may aware that, the elements and algorithm steps of each of the examples described in connection with the embodiments disclosed herein can be implemented in electronic hardware, or in combination with computer software and electronic hardware. Whether these functions are implemented by hardware or software depends on the specific application and design constraints of the technical solution. The skilled people could use different methods to implement the described functions for each particular application, however, such implementations should not be considered as going beyond the scope of the present application.

It should be understood that, in the embodiments of the present application, the disclosed device/terminal device and method could be implemented in other ways. For example, the device described above are merely illustrative; for example, the division of the units is only a logical function division, and other division could be used in the actual implementation, for example, multiple units or components could be combined or integrated into another system, or some features can be ignored, or not performed. In another aspect, the coupling or direct coupling or communicating connection shown or discussed could be an indirect, or a communicating connection through some interfaces, devices or units, which could be electrical, mechanical, or otherwise.

The units described as separate components could or could not be physically separate, the components shown as units could or could not be physical units, which can be located in one place, or can be distributed to multiple network elements. Parts or all of the elements could be selected according to the actual needs to achieve the object of the present embodiment.

In addition, the various functional units in each of the embodiments of the present application can be integrated into a single processing unit, or exist individually and physically, or two or more than two units are integrated into a single unit. The aforesaid integrated unit can either be achieved by hardware, or be achieved in the form of software functional units.

If the integrated unit is achieved in the form of software functional units, and is sold or used as an independent product, it can be stored in a computer readable storage medium. Based on this understanding, a whole or part of flow process of implementing the method in the aforesaid embodiments of the present application can also be accomplished by using computer program to instruct relevant hardware. When the computer program is executed by the processor, the steps in the various method embodiments described above can be implemented. Wherein, the computer program comprises computer program codes, which can be in the form of source code, object code, executable documents or some intermediate form, etc. The computer readable medium can include: any entity or device that can carry the computer program codes, recording medium, USB flash disk, mobile hard disk, hard disk, optical disk, computer storage device, ROM (Read-Only Memory), RAM (Random Access Memory), electrical carrier signal, telecommunication signal and software distribution medium, etc. It needs to be explained that, the contents contained in the computer readable medium can be added or reduced appropriately according to the requirement of legislation and patent practice in a judicial district, for example, in some judicial districts, according to legislation and patent practice, the computer readable medium doesn't include electrical carrier signal and telecommunication signal.

As stated above, the aforesaid embodiments are only intended to explain but not to limit the technical solutions of the present application. Although the present application has been explained in detail with reference to the above-described embodiments, it should be understood for the ordinary skilled one in the art that, the technical solutions described in each of the above-described embodiments can still be amended, or some technical features in the technical solutions can be replaced equivalently; these amendments or equivalent replacements, which won't make the essence of corresponding technical solution to be broken away from the spirit and the scope of the technical solution in various embodiments of the present application, should all be included in the protection scope of the present application.

What is claimed is:

1. A brainprint signal recognition method, comprising:
acquiring a brainprint signal to be classified, mapping the brainprint signal to be classified into a vector space, and determining a coefficient vector of the brainprint signal to be classified;
acquiring class centers and distance thresholds of respective existing classes in the vector space, wherein each of the existing classes corresponds to a brainprint signal set; and
determining, according to the coefficient vector of the brainprint signal to be classified and the class centers and the distance thresholds of the respective existing classes, the class to which the brainprint signal to be classified belongs.

2. The brainprint signal recognition method according to claim 1, wherein the step of determining, according to the coefficient vector of the brainprint signal to be classified and the class centers and the distance thresholds of the respective existing classes, the class to which the brainprint signal to be classified belongs comprises:
respectively calculating first distance values between the coefficient vector of the brainprint signal to be classified and the class centers of the respective existing classes, wherein each of the first distance values corresponds to a respective one of the existing classes;
determining, according to the respective first distance values and the distance thresholds of the corresponding existing classes, whether the brainprint signal to be classified belongs to a class in the existing classes; and
adding the brainprint signal to be classified into a brainprint signal set corresponding to a first class to obtain an updated brainprint signal set and re-determining a class center of the first class according to the updated brainprint signal set, in the case that the brainprint signal to be classified belongs to a class in the existing class, wherein the first class is the class to which the brainprint signal to be classified belongs.

3. The brainprint signal recognition method according to claim 2, wherein the step of determining, according to the respective first distance values and the distance thresholds of the corresponding existing classes, whether the brainprint signal to be classified belongs to a class in the existing classes comprises:
comparing any of the first distance values with the distance threshold of a respective one of the existing classes; and
determining that the brainprint signal to be classified belongs to the existing class corresponding to one of the first distance values in the case that the one of the first distance values is smaller than the distance threshold of a respective one of the existing classes.

4. The brainprint signal recognition method according to claim 2, wherein after the step of determining, according to the respective first distance values and the distance thresholds of the corresponding existing classes, whether the brainprint signal to be classified belongs to a class in the existing classes, the method further comprises:
creating an added class and determining a class center of the added class according to the coefficient vector of the brainprint signal to be classified in the case that the brainprint signal to be classified does not belong to a class in the existing classes.

5. The brainprint signal recognition method according to claim 4, wherein after the step of determining a class center of the added class according to the coefficient vector of the brainprint signal to be classified, the method further comprises:
acquiring second distance values between class centers of respective second classes, wherein the second class is a class in the updated classes composed of the added class and the existing classes; and
determining distance thresholds of the respective second classes according to the second distance values.

6. The brainprint signal recognition method according to claim 5, wherein the step of determining distance thresholds of the respective second classes according to the second distance values comprises:
calculating average distances between a class center of any of the second classes and class centers of the other second classes according to the second distance values, wherein each of the second classes corresponds to a respective one of the average distances;
taking a value smaller than a first preset threshold as the distance threshold of the any of the second classes in the case that the average distance corresponding to the any of the second classes is less than the first preset threshold; and
taking a value greater than a second preset threshold as the distance threshold of the any of the second classes in the case that the average distance corresponding to the any of the second classes is greater than the first preset threshold.

7. The brainprint signal recognition method according to claim 6, wherein the first preset threshold is an average value of the second distance values between the class centers of the respective second classes.

8. The brainprint signal recognition method according to claim 6, wherein each of the second classes corresponds to a second preset threshold, and the second preset threshold of the any of the second classes is a product of an average distance corresponding to the any of the second classes and a preset coefficient, wherein the preset coefficient is greater than 0 and less than or equal to 1.

9. The brainprint signal recognition method according to claim 1, wherein before the step of acquiring a brainprint signal to be classified and determining a coefficient vector of the brainprint signal to be classified in the vector space, the method further comprises:
acquiring brainprint signal sets corresponding to the respective existing classes and establishing the vector space, wherein each of the brainprint signal sets includes at least one brainprint signal sample;
mapping brainprint signal samples corresponding to the respective existing classes into the vector space and obtaining coefficient vectors corresponding to the respective brainprint signal samples; and
determining the class centers and the distance thresholds of the respective existing classes according to the coefficient vectors of the brainprint signal samples corresponding to the respective existing classes.

10. The brainprint signal recognition method according to claim 9, wherein the class center of any of the existing classes is an operation result of the coefficient vectors of the respective brain signal samples corresponding to the any of the existing classes.

11. The brainprint signal recognition method according to claim 9, wherein the class center of any of the existing classes is a vector with the smallest average distance to the coefficient vectors of the respective brain signal samples corresponding to the any of the existing classes in the vector space.

12. The brainprint signal recognition method according to claim 9, wherein the step of determining the class centers and the distance thresholds of the respective existing classes according to the coefficient vectors of the brainprint signal samples corresponding to the respective existing classes comprises:
determining the class centers of the respective existing classes according to the coefficient vectors of the brainprint signal samples corresponding to the respective existing classes;
calculating third distance values between the class centers of the respective existing classes; and
determining the distance thresholds of the respective existing classes according to the third distance values.

13. The brainprint signal recognition method according to claim 12, wherein the step of determining the distance thresholds of the respective existing classes according to the third distance values comprises:
calculating average distances between a class center of any of the existing classes and class centers of the other existing classes according to the third distance values, wherein each of the existing classes corresponds to a respective one of the average distances;
taking a value smaller than a fourth preset threshold as the distance threshold of the any of the existing classes in the case that the average distance corresponding to the any of the existing classes is less than a third preset threshold; and
taking a value greater than the fourth preset threshold as the distance threshold of the any of the existing classes in the case that the average distance corresponding to the any of the existing classes is greater than the third preset threshold.

14. A terminal device comprising a memory, a processor, and a computer program stored in the memory and operable in the processor, wherein the processor is configured to execute the computer program to implement steps of the method according to claim 1.

15. A computer readable storage medium with a computer program stored therein, wherein when the computer program is executed by a processor steps of the method according to claim 1 are implemented.
